(19) United States
(12) Patent Application Publication
(10) Pub. No.: US 2019/0015081 A1
(43) Pub. Date: Jan. 17, 2019

(54) Apparatus and Method for Offline Collection of Breath Samples for Nitric Oxide Measurement

(71) Applicant: Spirosure, Inc., Pleasanton, CA (US)

(72) Inventors: Solomon Ssenyange, Fremont, CA (US); David Anvar, Sunnyvale, CA (US); Brian Awabdy, Dublin, CA (US); Todd Smith, Sunnyvale, CA (US); Ryan Leard, Oakland, CA (US)

(73) Assignee: Spirosure, Inc., Pleasanton, CA (US)

(21) Appl. No.: 16/024,761

(22) Filed: Jun. 30, 2018

Related U.S. Application Data
(60) Provisional application No. 62/533,470, filed on Jul. 17, 2017.

(51) Int. Cl. A61B 10/00; A61B 5/097; A61M 16/20; A61B 5/08

(52) U.S. Cl. CPC A61B 10/00; A61B 5/097; A61B 2010/0087; A61B 5/082; A61M 16/208

(57) ABSTRACT

A breath collection and storage apparatus is disclosed for collecting and storing samples of exhaled breath for later analysis of nitric oxide contained in collected breath samples. The described apparatus provides for inhaling air into the lungs via a one-way air inflow portal through an airflow chamber via an inhalation/exhalation portal. Air inhaled into the lungs is then expelled back into the airflow chamber via the inhalation/exhalation portal and flowed into a breath storage vessel. A flow meter monitor, such as a flow meter or pressure gauge can be employed to monitor and control the rate of flow of the exhaled breath. A three-way valve can be incorporated into the air outflow portal to selectively permit discharge of exhaled breath to the outside or into the breath storage vessel. If desired, a programmable controller in electrical connection with the flow meter and three-way valve can be employed to maneuver the three-way valve to discharge and collection positions to allow for collecting and storing preselected portions of the exhaled breath. In addition, a flow rate restriction mechanism can be employed to automatically control the flow rate of exhaled air (breath) through the airflow chamber.

### CROSS REFERENCE TO RELATED APPLICATIONS

This application claims the benefit of U.S. Provisional Application No. 62/533,470 filed on Jul. 17, 2017, the subject matter of which is herein incorporated by reference in its entirety.

### FIELD OF THE INVENTION

The present invention relates generally to monitoring devices and related components used to measure pulmonary functions, and more particularly to testing for nitric oxide (NO), as well as other markers, associated with monitoring respiratory medical conditions.

### BACKGROUND

Respiratory diseases are some of the most common disorders in the world. Such respiratory diseases include conditions such as chronic obstructive pulmonary disease (COPD), asthma, cystic fibrosis and pulmonary fibrosis. COPD, for example, affects millions of people and is responsible for extensive morbidity and mortality in the United States. COPD is a term used to describe chronic lung diseases characterized by progressive development of airflow limitations that are usually not fully reversible with medication. The common symptoms of COPD include breathlessness, wheezing and a chronic cough.

Asthma is another example of a chronic lung disease with symptoms similar to COPD, such as breathlessness and wheezing, but etiologically distinct from COPD. Asthma is a prevalent health care problem; it affects millions in the United States and around the world. About 40% of patients with asthma can be classified as having moderate to severe asthma and would benefit from more frequent monitoring of their airway inflammation. Although COPD and asthma require different treatments, test results for COPD and asthma often overlap.

Asthma in particular is characterized by an inflammatory reaction in hyper-reactive airways that restrict airflow into the lungs. In recent years, measurement of exhaled nitric oxide (eNO) has been shown to be a non-invasive and complementary tool to other pulmonary function tests in assessing airway inflammation, specifically in subjects with asthma. Accordingly, the presence of eNO has become a well-known, globally accepted biomarker for airway inflammation.

Nitric oxide is produced endogenously in cells by NO synthase and secreted by eosinophils in the distal alveoli. Its production is increased in response to inflammatory cytokines (which is associated with asthmatic episodes), and exhaled NO is thought to be an indirect measurement of airway eosinophilic inflammation. Thus, nitric oxide exhaled from the lower airways (e.g., non-nasal airways) can be correlated with the degree of airway inflammation. Patients with asthma have high levels of NO in their exhaled breath. Nitric oxide levels increase prior to the presence of clinical symptoms and its levels decline in response to appropriate therapy as airway inflammation subsides. These two characteristics make this an ideal biomarker for managing asthma status. For this reason, in 2011, the American Thoracic Society (ATS) issued new guidelines recommending the measurement of exhaled nitric oxide for the diagnosis and management of asthma. A diagnosis of asthma can be made when the level of nitric oxide in exhaled breath exceeds 50 ppb. High eNO levels are also associated with other inflammatory respiratory conditions.

In diagnosing respiratory diseases, a series of tests for eNO may be conducted. For example, point-of-care breath analyzers can provide eNO information to a physician or in a clinical setting, while handheld or portable breath analyzers can provide exhaled nitric oxide information to an individual patient. Details regarding a respiratory monitor useful for the detection of eNO is described in U.S. Patent Publication No. 2015/0250408 A1, titled “Respiratory Monitor,” the entirety of which is incorporated by reference herein. Details regarding a respiratory monitor useful for the detection of eNO also are described in U.S. Patent Publication No. 2017/0065208 A1, titled “Respiratory Monitor,” the entirety of which also is incorporated by reference herein. Respiratory devices using other sensors and other technologies also may test for various other biomarkers in a patient's breath.

For patients attempting to provide physicians monitoring their respiratory conditions, it is not always possible to have access to real-time NO analysis. Under these circumstances, being able to collect one or more breath samples and store them for later analysis is beneficial to monitoring a patient's respiratory status. However, for any later analysis to be accurate and beneficial to the physician's treatment of their patients, the collection of breath samples must be consistent, and the storage of the sample must maintain the sample's integrity. For example, for patients collecting their own breath samples, patients must be able to collect and store the correct portion of their exhaled breath, exhale at the correct flow rate, and do so consistently.

Thus, it is desirable and advantageous to provide an apparatus that permits users to consistently and accurately capture exhaled breath samples and to properly store the collected breath samples for later nitric oxide analysis.

### BRIEF SUMMARY OF THE INVENTION

The present invention is generally directed to an apparatus and method for collecting and storing a breath sample for later nitric oxide measurement. In one embodiment, the apparatus comprises an airflow chamber in fluid communication with an inhalation/exhalation portal, as well as in fluid communication with a one-way air inflow portal and an air outflow portal. A flow meter, or pressure gauge, in fluid communication with the airflow chamber can be used to measure the airflow rate within the chamber. The air outflow portal is detachably connected to a breath storage vessel, such as a gas sample bag. Additionally, a filter, or scrubber, can be positioned upstream of the air inflow portal to substantially remove undesirable compounds, such as nitric oxide, during inhalation of air into the lungs. A desiccant may also be positioned upstream of the breath storage vessel to substantially reduce humidity in the breath sample being collected. In an alternate embodiment, the air inflow portal may be omitted where the user need only exhale air from the lungs into the airflow chamber for collection and storage of a breath sample.

In practice, in one embodiment, air is inhaled into the lungs through an airflow chamber via the inhalation/exhalation port via a one-way valve. The inhaled air is then exhaled through the inhalation/exhalation port back into the airflow chamber and into the breath storage vessel. In some embodiments, a three-way valve is placed in fluid communication between the airflow chamber and the breath storage vessel to allow discharge of exhaled breath to either the outside or into the breath storage vessel. In other embodiments, a programmable controller can be placed in electrical connection with the flow meter and the three-way valve to allow the automatic switching of the three-way valve from discharge of the exhaled breath to the outside or collection of the breath into the breath storage vessel, thereby allowing collection of preselected portions of the exhaled breath. In still other embodiments, the flow rate of exhaled breath through the airflow chamber can be controlled automatically through mechanisms that adjust the resistance to the rate of flow that are positioned downstream of the inhalation/exhalation portal to maintain the flow rate within certain parameters. Such mechanisms can include automated needle valves, automated adjustable apertures and the like.

### BRIEF DESCRIPTION OF THE SEVERAL VIEWS OF THE DRAWINGS

FIG. 1 illustrates schematically the inflow and outflow of air into and out of the airflow chamber according to one embodiment of the present invention.

FIG. 2 is a side view illustrating the airflow chamber, filter and one-way inflow components according to one embodiment of the present invention.

FIG. 3 is an exploded view of the apparatus illustrating one embodiment of the present invention

### DETAILED DESCRIPTION OF THE INVENTION

As used herein, the terms “comprises,” “comprising,” “includes,” “including,” “has,” “having,” or any other variation thereof, are intended to cover a non-exclusive inclusion. For example, processes, methods, articles, or apparatuses that comprise a list of elements are not necessarily limited to only those elements but may include other elements not expressly listed or inherent to such processes, methods, articles, or apparatuses. Further, unless expressly stated to the contrary, “or” refers to an inclusive “or” but not to an exclusive “or.” For example, a condition A or B is satisfied by any one of the following: A is true (or present) and B is false (or not present), A is false (or not present) and B is true (or present), and both A and B are true (or present).

Also, use of “a” or “an” are employed to describe the elements and components of the invention. This is done merely for convenience and to give a general sense of the invention. This description includes one or at least one and the singular also includes the plural unless it is obvious that it is meant otherwise.

Unless otherwise defined, technical and scientific terms used herein have the same meaning as commonly understood by one of ordinary skill in the art to which this invention belongs. Although methods that are similar or equivalent to those described herein can be used in the practice or testing of the present invention, suitable methods and materials are described herein. All publications, patent applications, patents, and other references mentioned herein are incorporated by reference in their entirety. In case of conflict, the present specification, including definitions, will control. In addition, materials, methods, and examples are illustrative only and not intended to be limiting.

In the following description, numerous specific details, such as the identification of various system components, are provided to understand the embodiments of the invention. One skilled in the art will recognize, however, that embodiments of the invention can be practiced without one or more of the specific details, ordinary methods, components, materials, etc. In still other instances, well-known structures, materials, or operations are not shown or described in detail to avoid obscuring aspects of various embodiments of the invention.

Reference throughout this specification to “one embodiment” or “an embodiment” means that a particular feature, structure, or characteristic described in connection with the embodiment is included in at least one embodiment of the present invention. Thus, the appearance of the phrases “in one embodiment” or “in an embodiment” in various places throughout this specification are not necessarily all referring to the same embodiment. Furthermore, the particular features, structures, or work characteristics may be combined in any suitable manner in one or more embodiments.

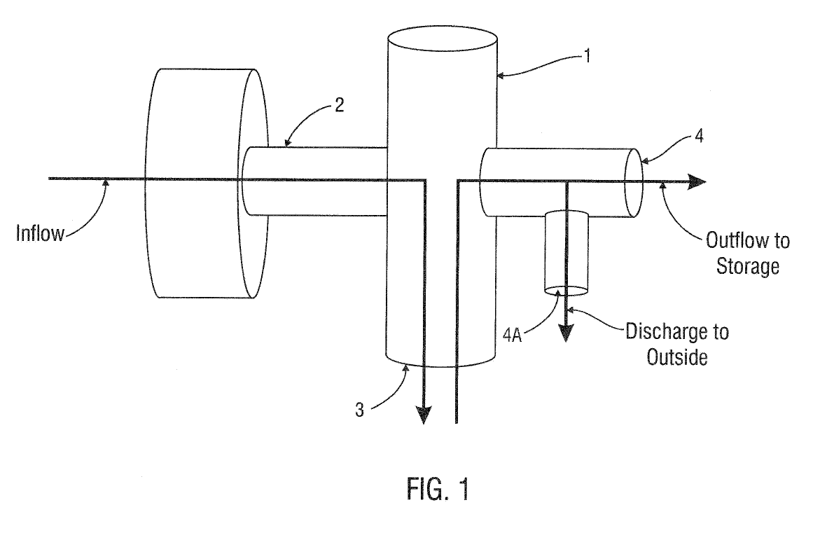

The present invention allows for collecting a sample of breath into a vessel for later nitric oxide analysis. Referring to FIG. 1, the airflow in one embodiment of the invention is illustrated schematically. Air is drawn into an airflow chamber 1 through a one-way air inflow portal 2 by inhalation through an inhalation/exhalation portal 3 that is in fluid communication with the inflow portal and the airflow chamber. The air that is inhaled into the lungs is then exhaled back into the airflow chamber through the same inhalation/exhalation portal. The one-way inflow portal prevents the exhaled air (breath) from exiting the airflow chamber via the inlet portal. Air that is expelled from the lungs flows to an air outflow portal 4 in fluid communication with the airflow chamber. The exhaled air is flowed out of the airflow chamber via the outflow portal and into a detachable breath storage vessel (not shown), or it may be discharged to the outside through discharge portal 4A. To prevent inflow of air through the outflow portal or the discharge portal during inhalation, a one-way check valve (not shown) is situated upstream of the outflow portal and discharge portal.

The airflow chamber, air inflow portal, inhalation/exhalation portal, air outflow portal, and discharge portal are dimensioned taking into account pressure, flow, and resistance factors to accommodate the ease of inhaling air through the apparatus and into the lungs, and then expelling the inhaled air from the lungs back into the airflow chamber and into the breath storage vessel. While it is desirable to maintain a low resistance during the inhalation process, the resistance during exhalation of breath from the mouth needs to be sufficient to close the velum. In most instances, the exhalation airflow resistance must be greater than five inches of water to close the velum.

In the below description, the air inflow portal and air outflow portal and the inhalation/exhalation portal are illustrated by reference to specific structures for purposes of describing the invention. Those of skill in the art will recognize alternatives to the specific structures described.

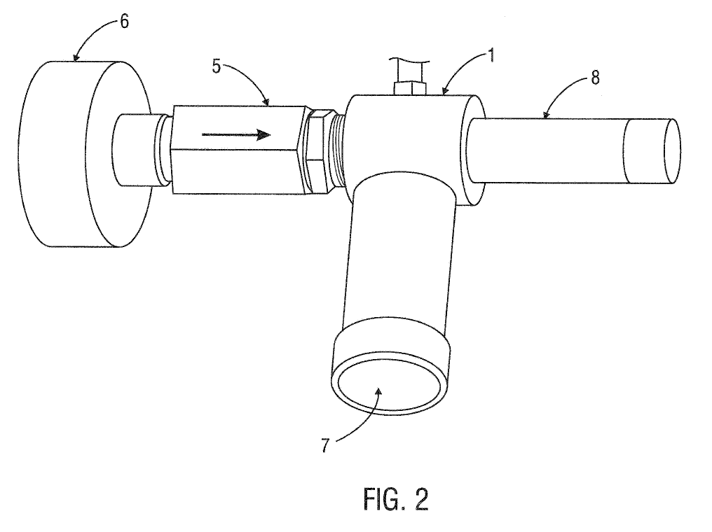

Referring to FIG. 2, airflow chamber 1 is shown in fluid communication with a one-way air inflow portal shown as one-way valve 5. The air inflow portal allows air to be inhaled into the airflow chamber, while preventing air exhaled into the airflow chamber from exiting to the outside via the air inflow portal. The apparatus preferably includes a filter, or scrubber, 6 positioned upstream of the one-way air inflow valve to reduce or substantially remove compounds that might interfere with the analysis of the collected breath sample, while preventing the exhaled breath from passing back through the filter. Such undesirable compounds can include, for example, nitric oxides, sulfur oxide, volatile organic compounds, particulates, and the like. An inhalation/exhalation portal attachment point 7 allows for attachment of a suitable device for inhaling and exhaling through the airflow chamber, such as, for example, a suitable mouthpiece or nosepiece depending on the type of nitric oxide analysis desired. Air that is expelled from the lungs into the airflow chamber is then flowed through air outflow conduit 8 towards a breath storage vessel. A one-way valve (not shown) is incorporated proximate to the air outflow conduit to prevent air from being pulled into the airflow chamber through the outflow conduit during breath inhalation.

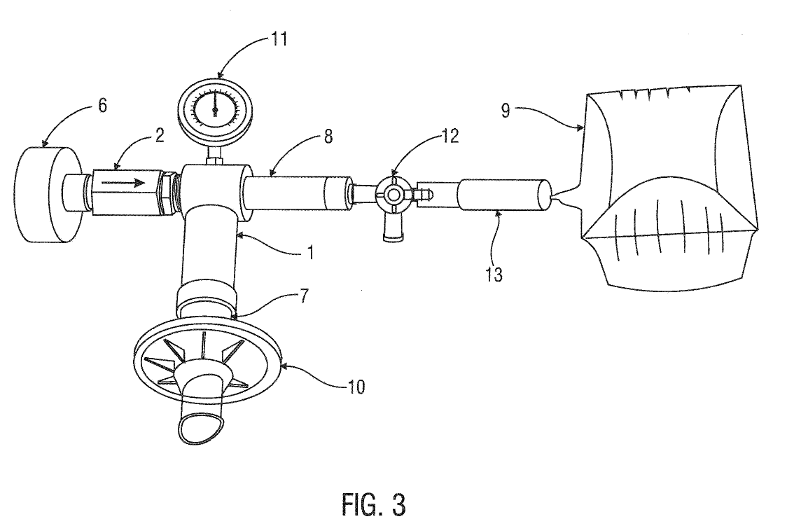

Referring more specifically to FIG. 3, air that is exhaled from the lungs into the airflow chamber flows via air chamber outflow conduit 8 and towards the breath storage vessel 9. Those of skill in the art will recognize suitable breath storage vessels for use in the present invention. Preferably the vessel is leak-proof and inert. An example of a suitable breath storage vessel is a Tedlar® gas sampling bag available from Millipore Sigma. When the breath sample is collected, the breath storage vessel is sealed. For later analysis for nitric oxide, the contents of the breath storage vessel can be pumped, or if a bag, squeezed into the nitric oxide analyzer, such as the respiratory monitor described in U.S. Patent Publication No. 2015/0250408 A1, which is hereby incorporated by reference in its entirety.

Again referring to FIG. 3, an inhalation/exhalation portal is shown as a mouthpiece 10 in fluid communication with the air chamber 1, which is attached to the airflow chamber inhalation/exhalation portal attachment point 7. A suitable detachable and disposable mouthpiece is, for example, the VBMax Standard PFT Filter P/N 156300 made by A-M Systems, which also provides relatively low resistance and bacterial and viral filtering.

In practice, it is desirable to control the flow rate of the exhaled air. Accordingly, feedback on the flow rate of exhaled breath can be monitored by an airflow monitor, such as a flow meter or pressure gauge 11. Flow rates can range from about one liter per minute to about six liters per minute. Preferred flow rates are in general three liters per minute (plus or minus 10%) or ideally between 2.7 liters per minute and 3.3 liters per minute.

In addition, in practice it is often desirable to collect a more desirable portion of the exhaled breath for analysis by discharging a portion of exhaled breath to the outside. Although the times can be adjusted to any amount for collecting the desired portion of breath, it is generally preferred to discharge an initial portion of the exhaled breath to the outside, then collect a subsequent portion in a breath collection vessel. These times most often range from three to seven seconds for discharging air to the outside before collecting the second portion of exhaled breath. For example, to collect a more preferred portion of the exhaled breath, it is often desirable to discard the first portion of the exhaled breath to the outside, e.g. through the discharge portal 4A, and route a second portion of the exhaled breath into the breath storage vessel 9. Referring to FIG. 3, a three-way valve 12 is shown as the air outflow portal. The three-way valve allows an initial portion of breath to be discharged to the outside, and then positioned to flow the breath into the breath storage vessel. For example, it is often desirable to discharge the first five seconds of the breath being exhaled by mouth to the outside, then switching the valve to collect the final five seconds (approximately 0.25 liters) of the air being exhaled into the breath storage vessel. In order to provide a larger sample size for more accurate nitric oxide analysis, the maneuver can be conducted more than once, e.g., two inhalations and exhalations to collect approximately 0.5 liters of exhaled breath in the breath storage vessel. If desired, humidity can be reduced in the collected breath sample by incorporating a desiccant upstream of the breath storage vessel, such as desiccant 13 illustrated as being disposed between the three-way valve 12 and the breath storage vessel 9. If the breath sample is being obtained from a nasal breath maneuver, the sample collection would be altered by shortening the discharge time of the first portion of the exhaled breath and recognizing that the overall breath exhalation maneuver would be shorter than an a mouth exhalation breath maneuver.

To facilitate the switching of the three-way valve from discharge to collection, the flow meter and the three-way valve can be electrically connected through a controller programmed to switch the three-way valve to discharge the exhaled breath to the outside. For example, the controller can be programmed to discharge exhaled breath to the outside, then switch the valve to direct the breath into the breath storage vessel. In a preferred embodiment, the controller can be programmed to discharge exhaled breath to the outside for approximately 3-7 seconds, then switch the valve to direct the breath into the breath storage vessel for a subsequent 3-7 seconds.

In addition, it is often desirable to automatically control the flow rate of exhaled breath through the airflow chamber to maintain the desired flow rate. This improves consistency as to the collected samples, and improves consistency in flow rates as between different users of the apparatus. Automatically controlling the flow rate through the airflow chamber can be achieved through mechanisms that adjust the resistance to the flow rate that are positioned downstream of the inhalation/exhalation portal to maintain the flow rate within certain parameters, e.g., a flow rate of approximately three liters per minute. Such mechanisms can include automated needle valves, automated adjustable apertures and the like. A programmable controller in electrical communication with a flow meter and the flow restriction mechanism can be employed to electrically control the flow rate through the airflow chamber.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with anyone or more of the features described herein. The breadth of the present invention is not to be limited by the subject specification, but rather only by the plain meaning of the claim terms employed.

This disclosure is sufficient to enable one of ordinary skill in the art to practice the invention, and provides the best mode of practicing the invention presently contemplated by the inventor. While a full and complete disclosure is made of specific embodiments of this invention, the invention is not limited by the exact construction, dimensional relationships, and operation shown and described. Various modifications, alternative constructions, design options, changes and equivalents will be readily apparent to those skilled in the art and may be employed, as suitable, without departing from the spirit and scope of the invention. Such changes might involve alternative materials, components, structural arrangements, sizes, shapes, forms, functions, operational features and the like.

What is claimed is:

1. An apparatus for collecting a breath sample, comprising:
(a) an exhalation portal in fluid communication with an airflow chamber;
(b) an air outflow portal in fluid communication with said airflow chamber;
(c) an airflow monitor in fluid communication with said airflow chamber; and
(d) a detachable breath storage vessel in fluid communication with said airflow chamber via said air outflow portal.

2. The apparatus of claim 1 wherein said air outflow portal is a three-way valve, said three-way valve maneuverable between an air discharge position and an air collection position.

3. The apparatus of claim 2 further comprising a programmable controller in electrical communication with said airflow monitor and said three-way valve, said programmable controller configured to control said valve position in response to readings from said airflow monitor.

4. The apparatus of claim 1 wherein said exhalation portal is a mouthpiece.

5. The apparatus of claim 1 wherein said exhalation portal is a nosepiece.

6. The apparatus of claim 1 further comprising a flow rate restriction mechanism positioned downstream of said exhalation portal.

7. An apparatus for collecting a breath sample, comprising:
(a) an inhalation/exhalation portal in fluid communication with an airflow chamber;
(b) a one-way air inflow portal in fluid communication with said airflow chamber;
(c) an air outflow portal in fluid communication with said airflow chamber;
(d) an airflow monitor in fluid communication with said airflow chamber; and
(e) a detachable breath storage vessel in fluid communication with said airflow chamber, said air outflow portal disposed between said airflow chamber and said breath storage vessel.

8. The apparatus of claim 7 wherein said inhalation/exhalation portal is a mouthpiece.

9. The apparatus of claim 7 wherein said inhalation/exhalation portal is a nosepiece.

10. The apparatus of claim 7 further comprising a scrubber disposed upstream of said one-way air inflow portal.

11. The apparatus of claim 7 wherein said air outflow portal is a three-way valve, said three-way valve maneuverable between an air discharge position and an air collection position.

12. The apparatus of claim 11 further comprising a programmable controller in electrical communication with said airflow monitor and said three-way valve, said programmable controller configured to control said valve position in response to readings from said airflow monitor.

13. The apparatus of claim 7 further comprising a flow rate restriction mechanism positioned downstream of said inhalation/exhalation portal.

14. An apparatus for collecting a breath sample, comprising:
(a) a mouthpiece in fluid communication with an airflow chamber;
(b) a one-way air inflow valve in fluid communication with said airflow chamber;
(c) a scrubber disposed upstream of and in fluid communication with said one-way air inflow valve;
(d) a three-way air outflow valve in fluid communication with said airflow chamber, said three-way valve maneuverable between an air discharge position and an air collection position;
(e) an airflow monitor in fluid communication with said airflow chamber;
(f) a detachable breath storage vessel in fluid communication with said airflow chamber, said three-way air outflow valve disposed between said airflow chamber and said breath storage vessel;
(g) a programmable controller in electrical communication with said airflow monitor and said three-way valve, said programmable controller configured to control said valve position in response to readings from said airflow monitor; and
(h) a flow rate restriction mechanism positioned downstream of said mouthpiece.

15. A method of collecting a breath sample, comprising:
(a) exhaling air from the lungs into an airflow chamber through an exhalation portal in fluid communication with said airflow chamber;
(b) monitoring the flow rate of said exhaled air through said airflow chamber;
(c) controlling the flow rate of said exhaled air through said airflow chamber to substantially one to six liters per minute; and
(d) collecting said exhaled air in a breath storage vessel that is detachably connected and in fluid communication with said airflow chamber.

16. The method of claim 15 further comprising discharging a first portion of air exhaled into said airflow chamber to the outside and collecting a second portion of air exhaled into said airflow chamber in said breath storage vessel.

17. The method of claim 16 wherein said first portion of air discharged is approximately the first three to seven seconds of air flowing through said airflow chamber and said second portion of air collected is approximately the next three to seven seconds of air flowing through said airflow chamber.

18. The method of claim 15 wherein said flow rate is controlled to between approximately 2.7 liters per minute and 3.3 liters per minute.

19. A method of collecting a breath sample, comprising:
(a) inhaling air into the lungs through a one-way air inflow portal and through an airflow chamber via an inhalation/exhalation portal in fluid communication with said airflow chamber and said one-way air inflow portal;
(b) exhaling said inhaled air through said inhalation/exhalation portal;
(c) monitoring the flow rate of said exhaled air through said airflow chamber;
(d) controlling the flow rate of said exhaled air through said airflow chamber to substantially one to six liters per minute; and
(e) collecting said exhaled air in a breath storage vessel that is detachably connected and in fluid communication with said airflow chamber.

20. The method of claim 19 further comprising discharging a first portion of air exhaled into said airflow chamber to the outside through a discharge portal and collecting a second portion of air exhaled into said airflow chamber into said breath storage vessel.

21. The method of claim 20 wherein said first portion of air discharged is approximately the first three to seven seconds of air flowing through said airflow chamber and said second portion of air collected is approximately the next three to seven seconds of air flowing through said airflow chamber.

22. The method of claim 19 wherein said flow rate is controlled to between approximately 2.7 liters per minute and 3.3 liters per minute.

23. The method of claim 19 comprising inhaling and exhaling said air drawn into said lungs into said breath storage vessel two or more times.
